(19) United States
(12) Patent Application Publication
(10) Pub. No.: US 2012/0253115 A1
(43) Pub. Date: Oct. 4, 2012

(54) ENDOSCOPIC SYSTEM

(76) Inventor: Edward Mark Erin, London (GB)

(21) Appl. No.: 13/496,867

(22) PCT Filed: Sep. 17, 2010
(86) PCT No.: PCT/EP2010/063742

§ 371 Date: Jun. 22, 2012

(30) Foreign Application Priority Data
Sep. 18, 2009 (GB) 0916374.2

(51) Int. Cl. A61B 1/018; A61B 1/267

(52) U.S. Cl. 600/104

(57) ABSTRACT

An endoscopic system comprises an endoscope having a fibreoptic cable bundle, an elongate insertion member for insertion into a patient and an operating port. Endoscopic tools, such as cytology brushes, may be inserted into the port. A piece of absorbent material is attached to a cytology brush which acts as a scaffold for the membrane allowing its controlled placement on internal body surfaces after it is inserted into the patient via the port and elongate insertion member. The material absorbs neat fluid from inside the patient. The cytology brush and SAM material are removed and the fluid extracted from the material. The SAM material is discarded, however the cytology brush may be reused on the same patient.

### CROSS-REFERENCE TO RELATED APPLICATION

This application is a national phase filing, under 35 U.S.C. §371(c), of International Application No. PCT/EP2010/063742, filed on Sep. 17, 2010, the disclosure of which is incorporated herein by reference in its entirety.

### FIELD OF THE INVENTION

This invention relates to an endoscopic system and particularly, although not exclusively, to a bronchoscopic system.

### BACKGROUND

Endoscopy is widely used as a diagnostic and clinical monitoring method for the visual inspection of the interior of the body, allowing tissue, cells and fluid samples to be removed for testing, as well as for minimally invasive surgery. An endoscope generally comprises a tube for insertion into a body cavity or small incision. The tube contains an optical system that conveys light from a light source into the body cavity and returns light to allow a practitioner to observe the interior of the cavity. A camera may be fitted to the tube. The optical system may be in the form of a fibre optic system, allowing the tube to be flexible. The tube may include a longitudinal passageway (or catheter insertion channel) to allow insertion of tools such as probes, brushes or like instruments into the cavity from the exterior of the patient.

### SUMMARY OF THE INVENTION

Bronchoscopy is a procedure which utilises a specific endoscope designed for insertion into the lung. There are several existing sampling techniques which attempt to detect robust biomarkers, seek accurate phenotyping of respiratory diseases and which can potentially track inflammatory changes in response to disease activity. Bronchoscopy is routinely performed on patients with respiratory disease in order to carry out bronchoalveolar lavage (BAL), endobronchial mucosal biopsy and brushings. BAL is the most common way in which to sample the components of the epithelial lining fluid (ELF) and to determine the inflammatory mediator composition of the pulmonary airways, and it is often used in immunological research as a means of sampling cells or pathogen levels in the lung. The procedure involves advancing a bronchoscope until it is wedged in a subsegmental bronchus at the desired location within the lung. Approximately 20 mL of saline is injected with a syringe via an operating port and longitudinal passageway of the bronchoscope. The flow of saline from the distal end of the bronchoscope is observed via the bronchoscope's optical system. Maintaining the wedge position, gentle suction is applied, collecting the lavage specimen in a collection trap, but at a high and unknown dilution. This process is repeated up to 5 times (with a total amount of introduced slaine of 100-120 mL) as needed to obtain an adequate specimen of about 40-60 mL. There is usually a 40-70% recovery of total instillate.

The unknown dilution and range in the volume of fluid retrieved can make the accurate evaluation of the severity or progress of a disease difficult and many sensitive markers of inflammation may remain below the limits of detection.

Another major clinical limitation for the utility of examining bronchoalveolar lavage fluid (BALf) is the large range of normal values for each parameter, which makes BALf insensitive in detecting disease. Furthermore, abnormalities in BALf are rarely specific for any of the lung diseases. There are some patients who have normal BALf constituents despite a definite disease and some without any evidence of disease despite abnormal BALf findings. There is large interindividual variation which may not be related to the disease, and the airspace cells and secretions may not reflect interstitial processes. Also, the removal of BALf may preferentially select, activate or injure some cells, and the composition of the epithelial lining fluid may change during the bronchoalveolar lavage.

Mucosal biopsy involves the removal of inner lung tissue fragments and bronchial brushing similarly involves the removal of endobronchial superficial cells. However, none of the existing techniques allow for accurate measurement of inflammatory mediators and biomarkers present in the lining fluid of the lung. Biomarkers and inflammatory mediators in the ELF reflect inflammation in the underlying tissue; hence it is important that they are accurately quantified.

Existing bronchoscopic procedures can have adverse effects including bleeding, infection or a reactive pyrexia.

According to the present invention there is provided an endoscopic system comprising: an elongate member for insertion into a body, the elongate member having a longitudinal passage; an elongate tool for insertion into the longitudinal passage; and a piece of absorbent material for attaching to the elongate tool for collecting a sample from inside the body and for subsequently removing the sample.

Such an endoscopic system allows undiluted and uncontaminated fluid to be removed from the body. The system is simple to construct and can be operated without any significant extra training by a physician with experience of endoscopy. The endoscopic system may include a bronchoscope. The operation of this bronchoscopic system can be performed during a routine bronchoscopy.

Preferably the piece of absorbent material is an absorptive matrix material having a high wicking rate and a high absorptive capacity such as a fibrous hydoxylated polyester absorptive matrix material. Such a material is less likely to cause damage, bleeding or other adverse effects within the body than existing techniques and can quickly obtain a sample of high volume.

According to the present invention there is provided a method of operating an endoscopic system comprising: inserting an elongate member into a body, the elongate member having a longitudinal passage; attaching a piece of absorbent material to an elongate tool; inserting the elongate tool into the longitudinal passage; and collecting a sample from inside the body with the absorbent material and subsequently removing the sample.

The endoscopic system may be a bronchoscopic system which allows other established and routine bronchoscopic procedures to be performed as normal following the inventive sampling method. The sample recovered can comprise undiluted lining fluids which will have improved signal to noise ratios and increased amounts of detectable inflammatory mediators compared with existing methods.

The invention also provides an absorbent sheet material for taking a sample of bodily fluid, the sheet material adapted to be configured into a structure suitable for attaching to an elongate tool for insertion into an endoscope. Preferably the sheet material is configured into a tubular structure such as a cylinder and held in this form by inert biomedical adhesive. It is suitable to supply such a sheet material separately from the other components of the system and in an individual, sterile packaging. The material is quick and easy to attach to the elongate tool and is a single use item which is discarded after use.

Preferably the absorbent sheet material will release the absorbed sample when subjected to a centrifuge process. Thus the material does not require any washing to extract the collected sample and neat secretions can be obtained.

### BRIEF DESCRIPTION OF THE DRAWINGS

Embodiments of the present invention will now be described, by way of example only, with reference to the accompanying drawings in which:

FIG. 1 illustrates an endoscope suitable for use in the present invention;

FIG. 2 illustrates a cytology brush suitable for use in the present invention;

FIG. 3 is a plan view of a piece of absorbent material with two strips of biomedical adhesive;

FIG. 4 illustrates the absorbent material of FIG. 3 formed into a cylinder;

FIG. 5A illustrates the absorbent material of FIGS. 3 and 4 attached to the cytology brush of FIG. 2;

FIG. 5B illustrates the absorbent material and brush arrangement of FIG. 5A housed inside a guide sheath of the cytology brush.

FIG. 5C illustrates the absorbent material and brush arrangement deployed from e guide sheath.

FIG. 6 is a flow chart describing the method of operation of an endoscopic system of the present invention.

### DETAILED DESCRIPTION

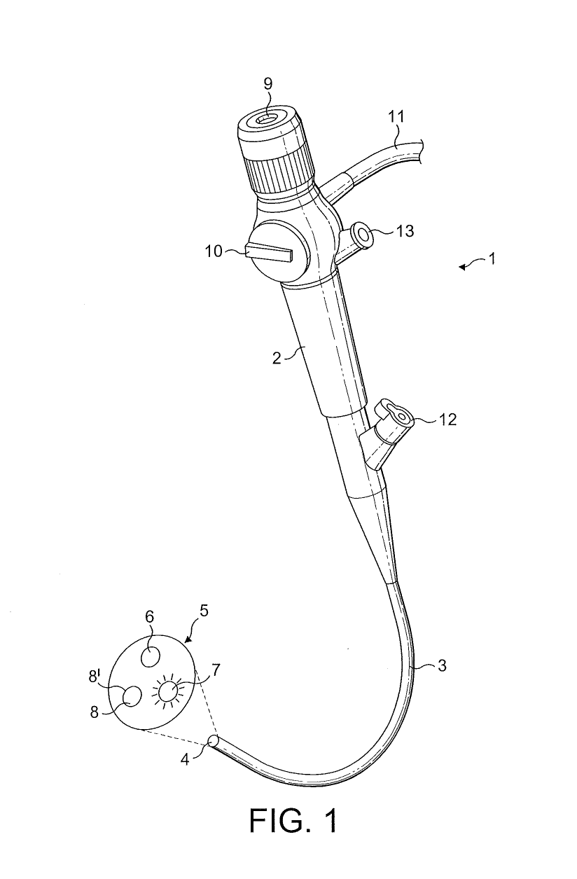

Referring firstly to FIG. 1, an endoscope 1 is illustrated comprising a housing 2 and an elongate insertion member 3 extending from the housing 2 and having a distal end 4 illustrated in a schematic, enlarged view 5. The insertion member 3 comprises a fibre optic cable bundle 6 that extends the length of the cord to allow the user to observe a field of view at the distal end 4 for example within a body cavity, a conduit that acts as light source 7 for the field of view under observation and an exit aperture 8′ of a channel 8 which extends longitudinally through the insertion member 3. The endoscope further comprises an eyepiece 9 located at the opposite end of the housing 2 from the insertion member 3 to allow the user to observe the fled of view at the distal end of the insertion member 3 through the fibre optic bundle 6. The housing 2 also has an associated control mechanism 10, input/output cable 11, an insertion channel port 12 and a suction channel port 13.

The insertion member 3 may be flexible or rigid or may have both rigid and flexible portions. The length of the insertion member 3 may be anything from a few centimetres to over 230 centimetres depending on the intended use.

The insertion channel port 12 is used for introducing and withdrawing sampling devices and fluid and for the introduction of medication. The channel 8 extends longitudinally through the insertion member 3 from the insertion channel port 12 to the exit aperture 8′. This channel branches inside the housing 2 such that it is also connected to the suction channel port 13. The suction channel port is configured to have a suction device attached to it and is used for removing fluid. The fibre optic cable bundle 6 extends between the distal end 4 of the insertion member 3 and the eyepiece 9. The light source conduit 7 is fed with light from an external source (not shown) through the input/output cable 11 The fibre optic cable bundle 6 transmits an image from the distal end 4 to the eyepiece 9, where it may be viewed by an operator of the endoscope 1. The image may also be output to a screen, recording unit or transmission means (not shown) through the input/output cable 11.

The control mechanism 10 allows the distal end portion 4 of the insertion member 3 to be dynamically bent and rotated. This is achieved via a system of longitudinally running Bowden cables that extend from within the member 3 near the distal end 4 to levers within the housing 2, forming part of the control mechanism 10. The flexible distal end of the member 3 allows the operator of the endoscope 1 to navigate the instrument and to change the view direction within a body cavity.

As well as providing the light source and an output for the fibre optic cable bundle 6. the input/output cable 11 may also provide electrical power to any other components of the endoscope requiring such power.

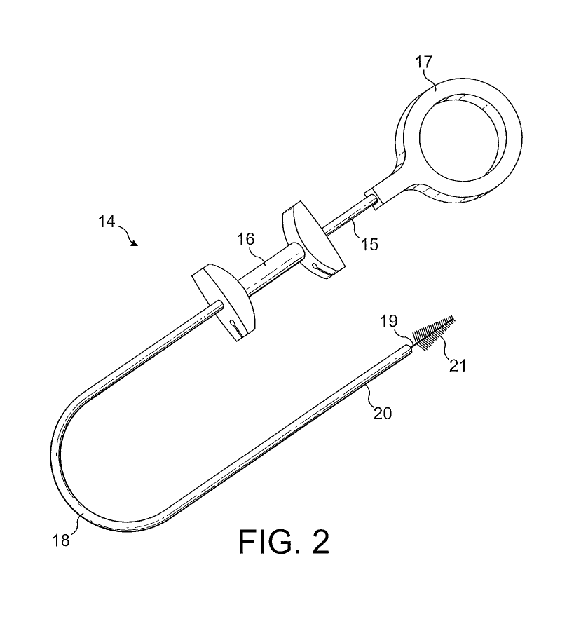

A tool which is often used during endoscopic procedures is a cytology brush and an example is shown in FIG. 2. The cytology brush 14 has a handle 15 comprising a grip portion 16, a ring portion 17 and a flexible elongate portion 18. The flexible elongate portion 18 is generally constructed of an inner wire 19 slidably received with a sheath 20 of plastics material. A brush portion 21 is located at the distal end of the cytology brush. The diameter of the inner wire 19 portion is 1 mm and the brush portion 21 diameter ranges from 1.2 mm to 5 mm depending on the intended use.

The ring portion 17 of the handle 15 is moveable with respect to the grip portion 16. When the ring portion 17 is pulled, it moves away from the grip portion 16 and causes the inner wire 19 to move within the sheath 20. This action causes the brush portion 21 to be retracted into the plastic sheath 20 of the flexible elongate portion 18. When the ring portion 17 is pushed back towards the grip portion 16, the brush portion 21 protrudes from the sheath 20.

The cytology brush 14 is designed to be inserted into the endoscope 1 through the insertion channel port 12 for example to perform a brushing within the lung to take a sample. The ability to retract and deploy the brush portion 21 facilitates the protection of any sample the brush has collected from contamination as the cytology brush 14 is withdrawn from the endoscope 1.

Preferably the sheath 20 has a 2.6 mm inner diameter channel and the endoscope insertion channel has an inner diameter of 2.8 mm.

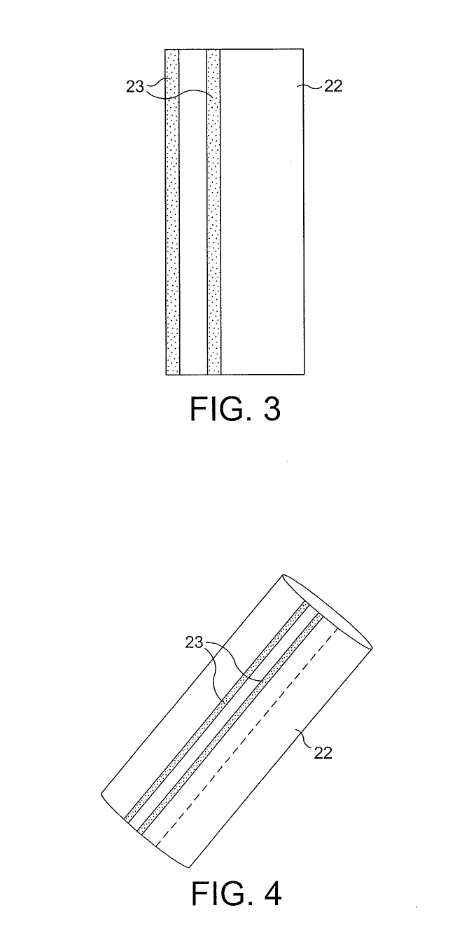

FIGS. 3 and 4 illustrate a piece of absorbent sheet material 22 such as an analytical membrane for use in the present invention. The material 22 is configured to be attached to or scaffolded over an endoscopic tool such as the cytology brush of FIG. 2. The piece of material 22 may be of any dimensions suitable for attachment to an endoscope tool. The piece may, for example, be approximately 7 mm wide and 50 mm long.

The material 22 may be any substance suitable for benign introduction into the human body and for absorbing fluid. The material 22 may be constructed from a number of quality controlled base materials, for example, graded 100% cellulose fibre, cellulose and rayon blend, borosilicate glass fiber with PVA binder, cellulose and synthetic blend with PVA binder or a fibrous hydoxylated polyester. The material 22 may be provided in various thicknesses, absorbencies and wick rates to meet the specific sampling needs. The piece of absorbent material 22 may preferably have a fast wicking rate (<20 s/3 cm) and a high absorption capacity (>100 μL/cm²) to allow for rapid absorption of a high volume of bronchial epithelial lining fluid.

An example of a material suitable for use in the present invention is “Accuwick Ultra”, manufactured by Pall Corporation (Europa House, Havant Street, Portsmouth, Hampshire, PO1 3PD). The material may be provided in a pre-sized, individual form as shown in FIG. 3 by Parafix Tapes & Conversions Ltd (Spencer Road, Lancing Business Park, Lancing, West Sussex. BN15 8UA). Alternatively, the material may be provided as several units which require manual detachment or may come as a roll of many units. The material may be further sterilised with gamma radiation after being attached to an endoscope tool. The individual material pieces may come in a sterile packaging for opening immediately prior to use.

The piece of absorbent material 22 may have an absorbent sink (not shown) located at one end of the material 22. This sink acts as a reservoir for the fluid sample after it has travelled through the material via a wicking process. The absorbent sink is typically constructed of either glass fibre or cellulose materials and helps to control the flow rate of fluid into the absorbent material 22. The absorbent sink preferably has the same thickness as the absorbent material 22, and is provided pre-fabricated with the absorbent material 22.

The absorbent material 22 has strips 23 of adhesive, for example a double sided inert sticking tape as manufactured by Parafix Tapes & Conversions Ltd. The adhesive may alternatively be an inert biomedical glue. The strips 23 of adhesive do not contain a residual solvent and are safe for introduction into the human body. The adhesive may be applied by a technician or physician after removing the material 22 from any packaging or may be pre-applied prior to any packaging of the material 22. The adhesive strips 23 may have a peel-off covering to prevent the strips sticking to any packaging. The adhesive substance may be arranged in one or more longitudinal strips 23 which may extend the entire length of the absorbent material 22, or over only a portion of its length. The adhesive substance may alternatively be arranged in one or more curved strips or in patches and may be located along one or both sides of the piece of absorbent material 14. Tests with the Accuwick Ultra absorptive matrix material have shown that a piece of the material of dimensions 7 mm by 50 mm can absorb in excess of 250 μl of fluid.

FIG. 4 shows the piece of absorbent sheet material 22 of FIG. 3 formed into a cylinder. The material is preferably formed into a cylinder manually by a medical technician or a physician. The dashed line illustrates the edge position of the side of the absorbent material 22 which does not include the adhesive strips 23 and which may be hidden from view when the cylinder is formed.

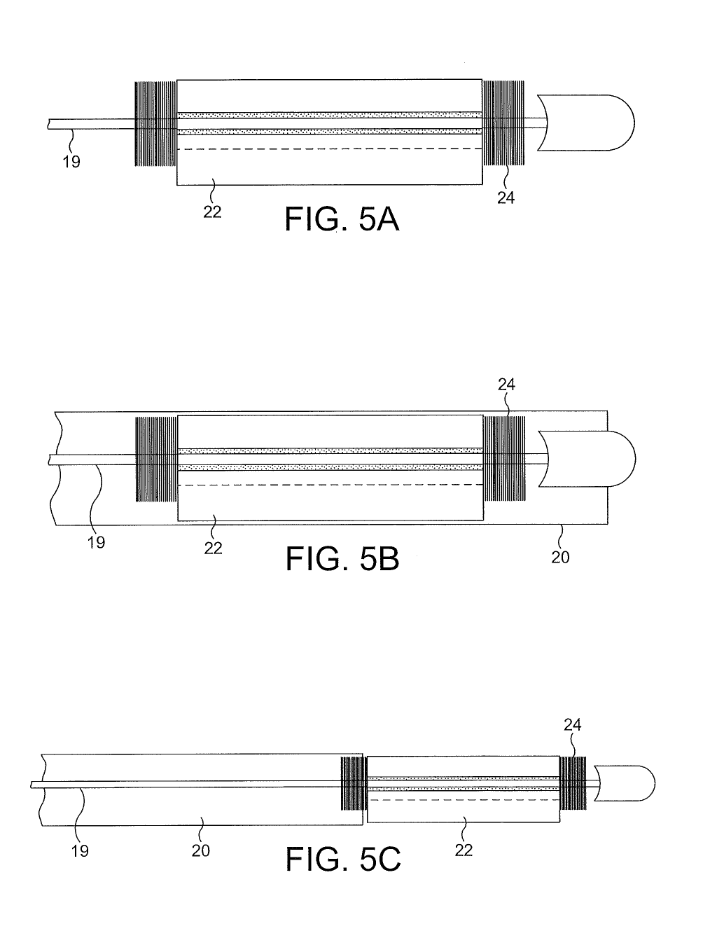

The absorbent material 22 is preferably formed into a cylinder around the brush portion 21 of the cytology brush as shown in FIG. 5A. The cylinder of absorbent material is affixed to the brush portion 21 by the friction between the bristles 24 and the inner surface of the cylinder. By forming the cylinder around the brush 21, a secure fit and strong attachment is provided. Preliminary tests have shown that a friction based attachment is sufficient to prevent detachment of the absorbent material 22 during an endoscopic procedure. However should the material become detached, it can be removed by endoscopic forceps.

FIG. 5B shows the absorbent material 22 formed into a cylinder around the brush portion 21, the brush portion 21 being located inside the sheath 20 of the elongate portion 18 of cytology brush 14. While in this position the elongate portion 18 of the cytology brush 14 is inserted into the endoscope 1 via the insertion channel port 12 without damaging the brush head or the affixed absorbent material 22 or dislodging the absorbent material 22.

FIG. 5C shows the brush portion 21 of the cytology brush 14 and affixed absorbent material 22 after being deployed from the sheath 20. While in this position the absorbent material 22 is able to collect a sample. The brush portion 21 is withdrawn into the sheath 20 in order to withdraw the cytology brush 14 from the endoscope 1.

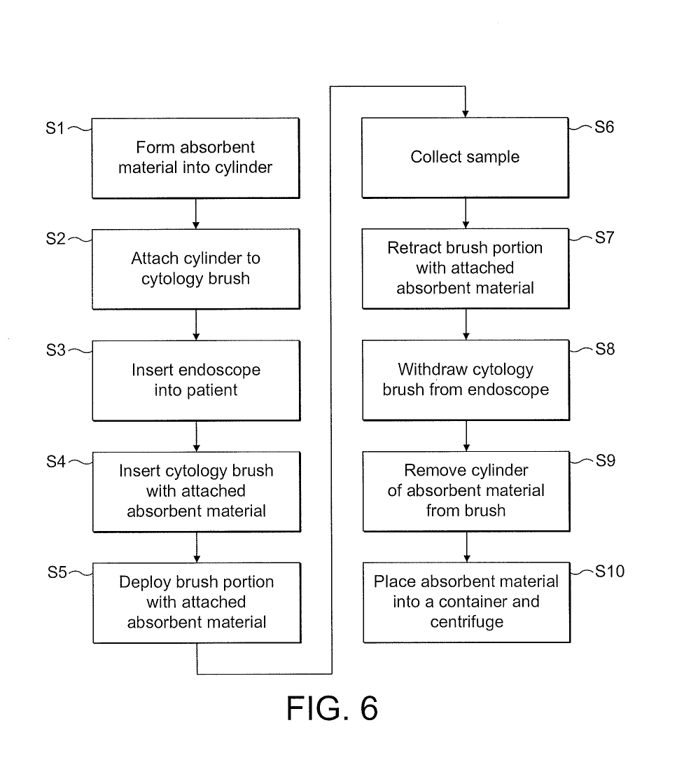

A preferred method of operating the endoscopic system will now be described with reference to FIG. 6. In step S1 the absorbent material 22 is formed into a cylinder as shown in FIG. 4 and in step S2 the cylinder of absorbent material is attached to the cytology brush 14. In practice these two steps may be performed simultaneously, with the absorbent material being fashioned around the brush portion 21 so that a secure fit results. In order to allow steps S1 and S2 to be performed, the brush portion 21 of cytology brush 14 is deployed from the sheath 20 by pushing the ring portion 17 of the handle 15 towards the grip portion 16. This exposes the brush portion 21 and allows the absorbent material 22 to be easily attached. Once the absorbent material 22 is attached to the brush portion 21, the brush portion 21 is retracted into the sheath 20.

At step S3 the insertion member 3 of the endoscope 1 is inserted into a body cavity. In bronschoscopy the elongate member is inserted through the nasal or oral cavity and down the trachea into the lung.

Once the endoscope has been inserted, the cytology brush 14 is inserted into the insertion channel port 12 at step S4. During insertion, the brush portion 21 remains inside the sheath 20 of the flexible elongate portion 18 so as not to cause contamination of the absorbent material.

The brush portion 21 with the absorbent material 22 attached is then deployed from the sheath 20 at step S5. This is achieved by the operator of the endoscope 1 pushing the ring portion 17 of the handle 15 towards the grip portion 16, causing the inner wire 19 to move within the sheath 20. The brush portion 21 need not necessarily be fully extended from the sheath 20, and some of the length of the absorbent material 22 may remain inside the sheath 20. The deployment of the brush portion 21 is observed by the endoscope operator through the eyepiece 9 or on a screen which the image is output to through the input/output cable 11. This allows the operator to carefully select the place within the body to which the brush portion 21 will be deployed and from which the absorbent material 22 will collect a sample. Such control is important to reduce the chance of the brush portion 21 causing damage.

A sample of fluid is absorbed by the absorbent material 22 at step S6. This is achieved by the absorbent material 22 coming into contact with an inner surface of the body cavity. The absorbent material 22 may typically be deployed for approximately 60 seconds.

Once a sample has been successfully collected, the brush portion 21 is retracted into the sheath 20 at step S7. This is achieved by the operator of the endoscope 1 pulling the ring portion 17 of the handle 15 away from the grip portion 16, causing the inner wire 19 to move within the sheath 20. This ensures that the absorbent material 22 does not become dislodged from the brush portion 21 as the brush is withdrawn and also prevents contamination of the sample. The cytology brush 14 may have a relatively large sheath of 2.6 mm inner diameter. This allows the absorbent material 22 to be easily accommodated within the sheath 20. The absorbent material 22 becomes engorged when it absorbs a sample of fluid and the large diameter sheath 20 ensures that the absorbent material 22 can be easily retracted while retaining a sample.

The cytology brush 14 is removed from the endoscope 1 at step S8. During this step the insertion member 3 of the endoscope 1 remains inside the body cavity. The endoscope operator pulls on the handle portion 15 of the cytology brush 14 to slide the elongate portion 18 out of the insertion channel of the endoscope 1.

At step S9 the absorbent material 22 is detached from the brush portion 21. In order to perform tests on the fluid sample, it is extracted from the absorbent material 22; this may be achieved by centrifuge.

At step S10 the absorbent material 22 is placed in a suitable container, such as an Eppendorf tube and then placed in a spin filter. Centrifugation is performed to obtain the neat fluid. The absorbent material 22 is preferably low protein binding in nature, allowing for an easy recovery of the protein mediators by centrifugation. Thus the absorbent material 22 does not require any elution or washing to extract the collected neat samples. The sample is therefore obtained in an undiluted form.

The absorbent material may be weighed at a time before step S1 and again after step S9. The increase in weight can then be compared with the volume of fluid collected. The piece of absorbent sheet material 22 is a single use item and should be discarded in a safe manner after use. The cytology brush may be used again during the same endoscopic procedure to collect cell samples; it is then discarded.

Preferably the method of the invention relates to a bronchoscope and bronchoscopic procedure. This method may be the sole procedure or may be performed in combination with other bronchoscopic procedures. Preferably the method described is the first procedure to be performed as it does not affect in any way the subsequent implementation of routine bronchoscopic procedures, such as endobronchial washing, brushing and biopsy. The undiluted fluid which is collected may be analysed using existing techniques to detect biomarkers. The neat samples obtained by this method may have greater than 10 times the level of detectable inflammatory mediators than samples obtained with existing procedures.

While the invention has been described with reference to a specific embodiment, variations be apparent to the person skilled in the art and these variations are intended to fall within the scope of the appended claims. For example, although the endoscopic system of the present invention has been described in terms of a bronchoscopic system, the invention may also be applied to thoroscopy, laparoscopy, nasendoscopy, colonoscopy, gastroscopy, cystoscopy and arthroscopy.

1. An endoscopic system comprising:
an elongate member for insertion into a body, the elongate member having a longitudinal passage;
an elongate tool for insertion into the longitudinal passage to extend from the elongate member for performing an endoscopic procedure and for subsequent withdrawal from the elongate member; and
a piece of absorbent material configured to be attached to the elongate tool for insertion to into the body through the longitudinal passage for collecting a sample from inside the body and for subsequently removing the sample from the body by withdrawal of the tool from the elongate member, wherein the piece of absorbent material is configured to be removed from the elongate tool after the withdrawal of the tool to provide the sample from inside the body.

2. An endoscopic system according to claim 1, wherein the endoscopic system is a bronchoscopic system.

3. An endoscopic system according to claim 1, wherein the elongate tool is a cytology brush.

4. An endoscopic system according to claim 1, wherein the absorbent material is an absorptive matrix material.

5. An endoscopic system according to claim 4, wherein the absorbent material is a matrix material having a high wicking rate and a high absorptive capacity.

6. An endoscopic system according to claim 1, wherein the absorbent material is configured to be formed into a cylinder and wrapped around the elongate tool.

7. An endoscopic system according to claim 6, wherein the absorbent material is configured to be secured in the form of a cylinder by inert biomedical adhesive disposed on the absorbent material.

8. An endoscopic system according to claim 1, wherein the sample is an undiluted bodily fluid.

9. An endoscopic system according to claim 8, wherein the undiluted bodily fluid is undiluted bronchial epithelial lining fluid.

10. An endoscopic system according to claim 1, wherein the absorbent material is configured to release the collected sample when subjected to a centrifuge process.

11. A method of operating an endoscopic system comprising:
inserting an elongate member into a body, the elongate member having a longitudinal passage;
attaching a piece of absorbent material to an elongate tool suitable for performing an endoscopic procedure;
inserting the elongate tool into the longitudinal passage of the elongate member;
collecting a sample from inside the body with the absorbent material and subsequently removing the sample from the body; and
subsequent to removing the sample from inside the body, removing the absorbent material from the elongate tool.

12. A method of operating an endoscopic system according to claim 11, wherein subsequently to removing the sample from the body, extracting the sample by subjecting the absorbent material to a centrifuge process.

13. An absorbent sheet material for taking a sample of bodily fluid, the sheet material configurable into a structure suitable for attaching to an elongate tool for insertion into an endoscope.

14. An absorbent sheet material according to claim 13, wherein the structure suitable for attaching to an elongate tool is a tubular structure.

15. An absorbent sheet material according to claim 13, wherein the sheet material is configurable into a structure by inert biomedical adhesive disposed on a part of the absorbent sheet material.

16. An absorbent sheet material according to claim 15, wherein the inert biomedical adhesive is disposed in one or more strips on the absorbent sheet material.

17. An absorbent sheet material according to claim 13, wherein the absorbent sheet material is a matrix material having a high wicking rate and a high absorptive capacity.

18. Apparatus for taking a sample of bodily fluid, the apparatus comprising an absorbent sheet material according to claim 13 and an elongate tool for insertion into an endoscope.

19. (canceled)

20. (canceled)

21. An endoscopic system comprising:
an elongate member for insertion into a body, the elongate member having a longitudinal passage;
a cytology brush for insertion into the longitudinal passage; and
a piece of absorbent sheet material, configured to be wrapped around the cytology brush, for collecting a sample from inside the body and for subsequently removing the sample.
